(19) United States
(12) Patent Application Publication
(10) Pub. No.: US 2023/0167444 A1
(43) Pub. Date: Jun. 1, 2023

(54) COMPOSITIONS AND METHODS FOR INTRANASAL TREATMENT WITH DOUBLE STRANDED RNA

(71) Applicant: THE TRUSTEES OF COLUMBIA UNIVERSITY IN THE CITY OF NEW YORK, New York, NY (US)

(72) Inventor: Carol M. TROY, Hastings-on-Hudson, NY (US)

(21) Appl. No.: 17/920,342

(22) PCT Filed: Apr. 21, 2021
(86) PCT No.: PCT/US2021/028457

§ 371 Date: Oct. 20, 2022

Related U.S. Application Data
(60) Provisional application No. 63/013,779, filed on Apr. 22, 2020.

(51) Int. Cl. C12N 15/113; A61P 25/00

(52) U.S. Cl. CPC C12N 15/113; A61P 25/00; C12N 2310/14

(57) ABSTRACT

The present disclosure relates to a method and composition for treating pulmonary and central nervous system disease which includes administering to patients with a pulmonary or central nervous system disease an effective amount of double stranded RNA. The effective amount of double stranded RNA may be conjugated to a cell-penetrating peptide. The double stranded RNA conjugated to a cell-penetrating peptide may be administered to a patient intranasally. The double stranded RNA may inhibit production of a target protein associated with symptoms of the disease.

### TECHNICAL FIELD

The present disclosure relates to methods and compositions for the intranasal treatment and inhibition of disease through use of a cell permeable RNA inhibitor containing double stranded RNA conjugated to a cell permeable peptide.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 210271_413WO_SEQUENCE_LISTING.txt. The text file is 3.6 KB, was created on Apr. 19, 2021, and is being submitted electronically via EFS-Web.

### BACKGROUND

Recently there has been a large number of biologics that have been approved for use or are in advanced clinical trials (Walsh. Nat. Biotechnol. (2006), 24, 769-776; Kumar et al., Curr. Pharm. Biotechnol. (2006), 7, 261-276; Jarvis et al., Chem. Eng. News (2009), 87, 28-29). Methods for their delivery are still primarily based on injectable formulations, often with inconvenient dosing regimens (Walsh, Nat. Biotechnol. (2006), 24, 769-776). For example, an important barrier to the ideal management of diabetes is a complex regimen of injections for insulin delivery, combining constant background (basal) release of insulin and increased dosages (bolus) after meals.

One of the biggest factors limiting the utility of double stranded RNA from becoming a more effective therapeutic is the inability to deliver double stranded RNA, like siRNA, to its intracellular target site due to their unfavorable physicochemical properties (negative charges, large molecule weight, and size) and instability, with plasma half-lives of about 10 min. Wang J, Lu Z, Wientjes M G, Au J L. Delivery of siRNA therapeutics: barriers and carriers. AAPS J. 2010; 12:492-503.

Accordingly, there is a need to provide a more convenient, less intrusive method of administering such therapeutic agents to patients, for example in the form of an effective intranasal delivery method molecular reservoir for which double stranded RNA may be delivered.

### SUMMARY

The present disclosure provides methods and compositions for the intranasal delivery of double stranded RNA including administering an effective amount of cell-permeable RNA inhibitor effective to treat a pulmonary or central nervous disease of a patient. The cell-permeable RNA inhibitor may include a double stranded RNA, which is effective in inhibiting the expression of the target protein, operably linked to a cell-penetrating peptide. The cell-penetrating peptide may be Penetratin1, transportan, pIS1, Tat(48-60), pVEC, MAP, Pep-1 or MTS. The cell-penetrating peptide may be linked to double stranded RNA by a disulfide bond. The concentration of the cell-permeable RNA inhibitor may be administered in a concentration between 1 nM and 1,000 nM, inclusive. The double stranded RNA may be siRNA, small temporal RNA, small nuclear RNA, small nucleolar RNA, short hairpin RNA or microRNA. Additionally, the double stranded RNA may be further attached to a label selected from the group including an enzymatic label, a chemical label, and a radioactive label

The double stranded RNA may be a p75NTR siRNA inhibitor, and the cell penetrating peptide may be Penetratin1. The concentration of the p75NTR siRNA inhibitor conjugated to the cell-penetrating peptide may be effective to treat the traumatic brain injury by decreasing apoptosis in the patient's brain. The concentration of the p75NTR siRNA inhibitor conjugated to the cell-penetrating peptide may also be effective to treat the traumatic brain injury by decreasing the amount of p75NTR in the patient's brain. This embodiment may be administered at a concentration between 1 nM and 500 nM, inclusive.

The present specification references various embodiments of the disclosure and provides various examples. These embodiments and examples may also be used in combination with one another and with any of the above methods unless they are clearly excluded therefrom.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure may be further understood through reference to the attached figures in combination with the detailed description that follows.

FIG. 1 is a pair of images that are low magnification images of the area of injury of a mouse who sustained a controlled cortical impact (“CCI”). Left image is stained for cleaved caspase-3 (“CC3”) a marker of dying cells, right image is the same section stained for p75NTR. Arrows show co-localization of p75NTR and CC3 positive cells.

FIG. 2 is a set of higher magnification images from mice that were either Sham (no injury) or mice subjected to CCI with brains harvested at 1 day or 3 days after CCI. Images were stained with terminal deoxynucleotidyl transferase deoxyuridine triphosphate nick end labeling (“TUNEL”), a marker of dying cells and anti-p75NTR.

FIG. 3A is a representative image of a western blot done on the olfactory bulb (“OB”) and cortex of mice that received either the p75NTR Pen1-siRNA inhibitor (“p75 si”) or a luciferase Pen1-siRNA control (“Ctrl si”) delivered intranasally.

FIG. 3B is a representative image of a western blot done on the OB, basal forebrain (“BFB”), striatum, and cortex of mice that received either the p75NTR Pen1-siRNA inhibitor (“p75 si”) or a luciferase Pen1-siRNA control (“Ctrl si”) delivered intranasally.

FIG. 4 is a graph of densitometry measures of p75NTR by Western blot done on the OB and cortex of mice from FIG. 3A. Values represent the means±SEM. Asterisks indicate significance by two-tailed, unpaired Student's t-test with p=0.04 for OB and p=0.01 for cortex.

FIG. 5 are representative images of cresyl violet-stained coronal sections from mice subjected to CCI followed by intranasal delivery of either luciferase Pen1-siRNA control (“ctrl siRNA”) or p75NTR Pen1-siRNA inhibitor (“p75 siRNA”), sections are marked with their coordinates to bregma.

FIG. 6 is graph of the difference in percent area of damage that mice developed if they were treated intranasally with a p75NTR Pen1-siRNA inhibitor (“p75NTR si”) or with luciferase Pen1-si control (“Ctrl si”). Percent area of damage is the region of tissue loss and penumbra as measured by cresyl violet-stained coronal sections. Values represent the means±SEM. Asterisks indicate significance by two-tailed, unpaired Student's t-test with p=0.03. n=3 mice/treatment.

FIG. 7 is a set of representative images of brain sections stained with neuronal nuclear antigen (“NeuN”) and counterstained with 4′,6-diamidino-2-phenylindole (“DAPI”) to reveal the area of damage one, three, or five days after treatment in mice subjected to CCI followed by intranasal delivery of either saline or p75NTR Pen1-siRNA inhibitor (“p75 siRNA”). Scale bar=200 μm.

FIG. 8 is a set of graphs of the area of damage in the micrographs shown in FIG. 7. For one day post injury p=0.065; for three days post injury p=0.03, for five days post injury, p0.028.

FIG. 9 is graph of the composite mNSS (“Modified Neurological Severity Score”) for naïve (no treatment), sham treated (everything except CCI), and mice subjected to CCI followed by treatment with intranasal luciferase Pen1-siRNA (“CsiR”), or p75NTR Pen1-siRNA (“siRp75”) mice.

FIG. 10 is a graph of hang test results, measured in seconds that mouse hung onto a rod, for naïve, sham treated, and mice subjected to CCI followed by treatment with intranasal luciferase Pen1-siRNA (“CsiR”), or p75NTR Pen1-siRNA (“siRp75”) mice.

FIG. 11 is a graph of horizontal ladder walking test results, measured in foot slips per run for naïve, sham treated, and mice subjected to CCI followed by treatment with intranasal luciferase Pen1-siRNA (“CsiR”), or p75NTR Pen1-siRNA (“siRp75”) mice. CL stands for limbs contralateral to injury.

FIG. 12 is a graph of the beam walking test results, measured in foot slips per run for naive, sham treated, and mice subjected to CCI followed by treatment with intranasal luciferase Pen1-siRNA (“CsiR”), or p75NTR Pen1-siRNA (“siRp75”) mice. CL stands for limbs contralateral to injury.

FIG. 13 is set of graphs of the composite mNSS for mice subjected to CCI followed by treatment with p75NTR Pen1-siRNA (siRNA) or saline.

FIG. 14 is a set of micrographs of astrocytes stained with sections stained for glial fibrillary acidic protein (“GFAP”) (green) or pro-brain-derived neurotrophic factor (“proBDNF”) (red). (a) Arrows indicate colocalization of proBDNF and GFAP-positive cells. (b) Arrowheads indicate GFAP-positive cells that do not express proBDNF. Scale bar=50 μm.

FIG. 15 is a graph of the composite mNSS for mice subjected to CCI followed by treatment with intranasal neutralizing antibodies to pro Nerve Growth Factor (“proNGF”) or proBDNF. Asterisks indicate significant difference from IgG control by Kruskal-Wallis test followed by Dunn's multiple comparison test for nonparametric values, with p=0.0204 for IgG control versus proNGF-treated mice; p=0.045 for IgG control versus proBDNF-treated mice.

FIG. 16 is a set of representative images of brain sections stained with NeuN and DAPI to reveal the area of damage three days after treatment in mice subjected to CCI followed by intranasal delivery of either anti proNGF or anti proBDNF antibodies or a control antibody (IgGRb) Scale bar=200 μm.

FIG. 17 is a set of graphs quantifying the area of tissue loss (p=0.006) or the area of total damage (p=0.016) in the micrographs of FIG. 16. The graphs depict the means±SEM. Asterisks indicate significance by one-way analysis of variance followed by Tukey's post hoc analysis with p<0.05.

FIG. 18 is a set of images from mice used for FIG. 16. Images were stained with TUNEL. Scale bar=50 μm.

FIG. 19 is a graph of TUNEL stained cells versus DAPI stained cells based on data from 3 to 4 animals per group from the cohort used to generate FIG. 16. p=0.02. The graph depicts the means t SEM. Asterisks indicate significance by one-way analysis of variance followed by Tukey's post hoc analysis with p<0.05.

### DETAILED DESCRIPTION

The present disclosure relates to methods for delivery of double-stranded RNA to a patient. For example, the present disclosure relates to a method for inhibiting p75 neurotrophin receptor (“p75NTR”) signaling activity associated with a traumatic brain injury by intranasally delivering double stranded RNA, such as siRNA, to a patient.

As used herein, the term “patient” refers to any animal, including any mammal, including, but not limited to, humans, and non-human animals including, but not limited to, non-human primates, dogs, cats, rodents, horses, cows, pigs, mice, rats, hamsters, rabbits, and the like. In particular, the patient is a human.

As used herein, an “effective amount” is an amount sufficient to cause a beneficial or desired clinical result in a patient. An effective amount may be administered to a patient in one or more doses. It is typically administered intranasally to the patient. In terms of treatment, an effective amount is an amount that is sufficient to ameliorate the impact of and/or inhibit the induction and/or exacerbation of traumatic brain injury in a patient, or otherwise reduce the pathological consequences of the infliction(s). The effective amount is generally determined by the physician on a case-by-case basis and is within the skill of one in the art. Several factors may be taken into account when determining an appropriate dosage to achieve an effective amount. These factors include age, the condition being treated, the severity of the condition, prior responses, type of inhibitor used, the pathway to be inhibited, the cell type expressing the target, and the form and effective concentration of the composition (also referred to herein as a “treatment,” “inhibitor,” or “conjugate”) being administered.

As used herein, “treat,” “treating” and similar verbs refer to ameliorating the impact of and/or inhibiting the induction and/or exacerbation of a disease or infliction in a patient. In some embodiments, the disease or infliction is traumatic brain injury.

### Method of Treating Traumatic Brain Injury and Other Diseases

In some embodiments, the instant disclosure is directed to methods of or uses of treatments disclosed herein in ameliorating the impact of and/or inhibiting the induction and/or exacerbation of diseases, such as traumatic brain injury, in a patient by administering an effective amount of cell permeable RNA inhibitor or conjugate thereof. In certain embodiments, the methods of the present disclosure are directed to the administration of a cell permeable RNA inhibitor, or conjugate thereof, via intranasal formulations in order to inhibit the negative symptoms of a disease, such as traumatic brain injury.

As used herein, the term “disease” refers to a clinically delectable ailment, dysfunction, or infliction. In some embodiments, the disease is a pulmonary or central nervous system, disease, dysfunction or infliction. As used herein, the term “traumatic brain injury” refers to clinically detectable brain dysfunction commonly caused by a physical blow to the head area. Traumatic brain injuries may also be caused by concussion, contusion, diffuse axonal injury, traumatic subarachnoid hemorrhage, and hematoma. Traumatic brain injury is also sometimes referred to as “craniocerebral trauma.” Clinical symptoms of traumatic brain injury may include confusion, blurry vision, and concentration difficulty. Clinical symptoms of traumatic brain injury may also include any of the following: irritability, reduction in cognitive function, memory loss, fatigue, headaches, visual problems, poor attention, sleep disturbances, seizures, vomiting, and feelings of depression.

In some embodiments, the cell permeable RNA inhibitor treats a disease by inhibiting expression of a target protein. In some embodiments, the target protein inhibits apoptosis (thereby producing a pro-apoptotic effect). In some embodiments, the target protein induces apoptosis (thereby producing an antiapoptotic effect). Reduction of target protein expression as a result of administration of cell permeable RNA inhibitor may be by at least about 10 percent, by at least about 20 percent, by at least about 30 percent, by at least about 40 percent, by at least about 50 percent, by at least 60 percent, by at least 70 percent, by at least 80 percent, by at least 90 percent, by at least 95 percent, or by between any of these percentages (e.g. by between 10 percent and 90 percent, by between 20 percent and 95 percent, by between 40 percent and 60 percent, etc.). In some embodiments, reduction of target protein expression treats a disease. In some embodiments, reduction of target protein expression reduces the symptoms of a disease.

The treatment, when used to treat the effects of a disease, may be administered as a single dose or multiple doses. For example, but not byway of limitation, where multiple doses are administered, they may be administered at intervals of 6 times per 24 hours or 4 times per 24 hours or 3 times per 24 hours or 2 times per 24 hours or 1 time per 24 hours or 1 time every other day or 1 time every 3 days or 1 time every 4 days or 1 time per week, or 2 times per week, or 3 times per week. In some embodiments, the initial dose may be greater than subsequent doses or all doses may be the same.

In some embodiments, the cell permeable RNA inhibitor used in connection with the methods and uses of the instant disclosure is a p75NTR siRNA inhibitor conjugate as disclosed herein. In some embodiments, the p75NTR siRNA inhibitor (or conjugate thereof) is administered to a patient suffering from traumatic brain injury either as a single dose or in multiple doses. The concentration of the cell permeable RNA inhibitor composition administered is, in some embodiments: 0.1 nM to 1,000 nM; 1 nM to 500 nM; 10 nM to 200 nM; or 60 nM to 100 nM, inclusive. In certain embodiments, a specific human equivalent dosage may be calculated from animal studies via body surface area comparisons.

In some embodiments, the cell permeable RNA inhibitor, either alone or in the context of a membrane-permeable conjugate, is administered in conjunction with one or more additional therapeutics. In some embodiments, the additional therapeutics include, but are not limited to a steroidal therapeutic. In some embodiments, the method involves the administration of one or more additional inhibitors either alone or in the context of a membrane-permeable conjugate. As used herein, “p75NTR inhibitor” refers to any drug, biologic, or molecule that inhibits the p75NTR signaling pathway. This may include p75NTR siRNA inhibitors or a p75NTR antagonist, like LM11a-3 or EVT901. In some embodiments, the p75NTR inhibitor is a siRNA.

In certain embodiments, the p75NTR inhibitor may treat traumatic brain injury by decreasing the amount of p75NTR in the brain of a patient. In certain embodiments, the p75NTR inhibitor may treat traumatic brain injury by decreasing apoptosis in the brain of the patient, as detected by a computed tomography scan or other brain scanning devices.

In further embodiments, the methods of treatment with cell permeable RNA inhibitor are adapted for use with other siRNAs, or double stranded DNA, and other cell penetrating peptides. In certain embodiments, a specific human equivalent dosage may be calculated from animal studies via body surface area comparisons. Many qualities of the treatment, such as, frequency of dosage, effective amount, selection of a proper double-stranded RNA, and selection of a proper cell-penetrating peptide may be selected based on the disease that is being treated and/or the patient's genotype and phenotype.

Double Stranded RNA

In some embodiments, the cell permeable RNA inhibitor described herein includes a double-stranded ribonucleic acid molecule operably linked to a cell-penetrating peptide. In some embodiments the double-stranded RNA is an inhibitor.

In some embodiments, a “double stranded ribonucleic acid molecule,” “double-stranded RNA.” or “dsRNA” refers to any RNA molecule including a double stranded portion, (e.g., containing an RNA duplex), notwithstanding the presence of single stranded gaps or overhangs of unpaired nucleotides. Further, as used herein, a double-stranded ribonucleic acid molecule includes single stranded RNA molecules forming functional stem-loop structures, such as small temporal RNAs, short hairpin RNAs and microRNAs, thereby forming the structural equivalent of an RNA duplex with single strand over-hangs. The RNA molecule of the present invention may be isolated, purified, native or recombinant, and may be modified by the addition, deletion, substitution and/or alteration of one or more nucleotides, including non-naturally occurring nucleotides or deoxyribonucleotides, including those added at 5′ and/or 3′ ends to increase nuclease resistance.

The double-stranded ribonucleic acid molecule of the cell permeable RNA inhibitor may be any one of a number of non-coding RNAs (i.e., RNA which is not mRNA, tRNA or rRNA), including a small interfering RNA (“siRNA”), but may also include a small temporal RNA, small nuclear RNA, small nucleolar RNA, short hairpin RNA or a microRNA including a double-stranded structure and/or a stem loop configuration including an RNA duplex with or without one or more single strand overhang. The double stranded RNA molecule may be very large, including thousands of nucleotides, or in the case of RNAi protocols involving mammalian cells, may be small, in the range of 21-25 nucleotides. In the dsRNA molecules of the invention, at least one strand includes a portion homologous to the target gene, where said homologous portion is between about 5 and 50, 10 and 30, or 15 and 28 nucleotides in length. In some embodiments, dsRNA of the present invention includes a double-stranded RNA duplex of at least 19 nucleotides including a 21 nucleotide sense and a 21 nucleotide antisense strand paired so as to have a 19 nucleotide duplex region and a 2 nucleotide overhang at each of the 5′ and 3′ ends. The 2 nucleotide 3′ overhang may include 2′ deoxynucleotides, e.g., TT, for improved nuclease resistance. In some embodiments, the double-stranded RNA is siRNA.

As used herein, “homologous” refers to a nucleotide sequence that has at least 80% sequence identity, the sequence may have at least 90%, at least 95%, or at least 98% sequence identity, or 100% sequence identity, to a portion of mRNA transcribed from the target gene. Homology may be determined using standard software such as BLAST or FASTA.

P75NTR siRNA Inhibitor

As used herein, a “p75NTR siRNA inhibitor” refers to siRNA that inhibits the p75NTR pathway.

In some embodiments, the p75NTR siRNA inhibitor has the sequence:

SSUGGAACAGCUGCAAACAAAUU (SEQ ID NO. 1).

p75NTR siRNA inhibitors include those sequences that retain certain structural and functional features of the above-identified p75NTR siRNA inhibitor yet differ from the identified inhibitor's sequence at one or more position, for example, p75NRT si RNA inhibitors may include variant dsRNAs as described herein.

In some embodiments, the p75NTR siRNA may be any sequence able to hybridize under stringent conditions or conditions that mimic the cellular environment to mRNA complementary to SEQ ID NO 1.

In some embodiments, the p75NTR siRNA inhibitor is attached to a conjugate.

Inhibitor-Cell Penetrating Peptide Conjugates

In some embodiments of the present disclosure, the double stranded RNA is conjugated to a cell penetrating peptide, typically via a disulfide bond, to form an inhibitor-cell penetrating peptide conjugate, herein referred to as a “cell permeable RNA inhibitor.” In some embodiments, the double-stranded RNA is siRNA.

As used herein, a “cell-penetrating peptide” is a peptide that includes a short (about 12-30 residues) amino acid sequence or functional motif that confers the energy-independent (i.e., non-endocytotic) translocation properties associated with transport of the membrane-permeable complex across the plasma and/or nuclear membranes of a cell. In some embodiments, the cell-penetrating peptide used in the membrane-permeable complex of the present disclosure include at least one non-functional cysteine residue, which is either free or derivatized to form a disulfide link with the double stranded RNA, which has been modified for such linkage. Representative amino acid motifs conferring such properties are listed in U.S. Pat. No. 6,348,185, the contents of which are expressly incorporated herein by reference. The cell-penetrating peptides of the present disclosure may include, but are not limited to, Penetratin1, transportan, pIs1, TAT(48-60), pVEC, MTS, MA and Pep-1. The cell-penetrating peptides of the present disclosure include those sequences that retain certain structural and functional features of the identified cell-penetrating peptides, yet differ from the identified peptides' amino acid sequences at one or more positions. Such polypeptide variants may be prepared by substituting, deleting, or adding amino acid residues from the original sequences via methods known in the art. In some embodiments, the cell-penetrating peptide is Penetratin1.

In certain embodiments, such substantially similar sequences include sequences that incorporate conservative amino acid substitutions. In some embodiments, a cell-penetrating peptide of the present disclosure is at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% homologous to the amino acid sequence of the identified peptide and is capable of mediating cell penetration. The effect of the amino acid substitutions on the ability of the synthesized peptide to mediate cell penetration may be tested using the methods disclosed in Examples section, below.

In certain embodiments of the present disclosure, the cell-penetrating peptide of the membrane-permeable complex is Penetratin1, including the peptide sequence C(NPys)-RQIKIWFQNRRMKWKK (SEQ ID NO: 2), or a conservative variant thereof. As used herein, a “conservative variant” is a peptide having one or more amino acid substitutions, wherein the substitutions do not adversely affect the shape—or, therefore, the biological activity (i.e., transport activity) or membrane toxicity—of the cell-penetrating peptide.

Other non-limiting embodiments of the present disclosure involve the use of the following exemplary cell permeant molecules: RL16 (H-RRLRRLLRRLLRRLRR-OH) (SEQ ID NO: 3), a sequence derived from Penetratin1 with slightly different physical properties; and RVG-RRRRRRRRR (SEQ ID NO: 4), a rabies virus sequence which targets neurons.

In certain alternative non-limiting embodiments of the present disclosure, the cell-penetrating peptide of the membrane-permeable complex is a cell-penetrating peptide selected from the group including: transportan, pIS1, Tat(48-60), pVEC, MAP, Pep-1 and MTS. Transportan is a 27-amino-acid long peptide containing 12 functional amino acids from the amino terminus of the neuropeptide galanin, and the 14-residue sequence of mastoparan in the carboxyl terminus, connected by a lysine. It includes the amino acid sequence GWTLNSAGYLLGKINLKALAALAKKIL (SEQ ID NO: 5), or a conservative variant thereof.

pIs1 is derived from the third helix of the homeodomain of the rat insulin 1 gene enhancer protein. pIs1 includes the amino acid sequence PVIRVW FQNKRCKDKK (SEQ ID NO: 6), or a conservative variant thereof.

Tat is a transcription activating factor, of 86-102 amino acids, that allows translocation across the plasma membrane of an HIV-infected cell, to transactivate the viral genome. A small Tat fragment, extending from residues 48-60, has been determined to be responsible for nuclear import; it includes the amino acid sequence GRKKRRQRRRPPQ (SEQ ID NO: 7), or a conservative variant thereof.

pVEC is an 18-amino-acid-long peptide derived from the murine sequence of the cell-adhesion molecule, vascular endothelial cadherin, extending from amino acid 615-632. pVEC includes the amino acid sequence LLIILRRRIRKQAHAH (SEQ ID NO: 8), or a conservative variant thereof.

MTSs, or membrane translocating sequences, are those portions of certain peptides which are recognized by the acceptor proteins that are responsible for directing nascent translation products into the appropriate cellular organelles for further processing. An MTS of particular relevance is MPS peptide, a chimera of the hydrophobic terminal domain of the viral gp41 protein and the nuclear localization signal from simian virus 40 large antigen; it represents one combination of a nuclear localization signal and a membrane translocation sequence that is internalized independent of temperature, and functions as a carrier for oligonucleotides. MPS includes the amino acid sequence GALFLGWLGAAGSTMGAWSQPKKKRKV (SEQ ID NO: 9), or a conservative variant thereof.

Model amphipathic peptides, or MAPs, form a group of peptides that have, as their essential features, helical amphipathicity and a length of at least four complete helical turns. An exemplary MAP includes the amino acid sequence KLALKLALKALKAALKLA (SEQ ID NO: 10)-amide, or a conservative variant thereof. Another relevant amphipathic peptide is Pep-1 with the sequence KETWWETWWTEWSQPKKKRKV (SEQ ID NO: 11).

Pep-1, or Pep-1 peptide, is a cell penetrating peptide that is a short amphipathic peptide with a hydrophobic tryptophan-rich domain and a hydrophilic lysine-rich domain separated by a spacer. Pep-1 includes the amino acid sequence KETWWETWWTEWSQ-PKKKRKV (SEQ ID NO: 12), or a conservative variant thereof.

In some embodiments the double-stranded RNA may be operably linked to the cell-penetrating peptide via a non-covalent linkage. In certain embodiments such non-covalent linkage is mediated by ionic interactions, hydrophobic interactions, hydrogen bonds, or van der Waals forces. In certain preferred embodiments, siRNA is linked to a cell-penetrating peptide. Certain embodiments, may require protection of the double-stranded RNA's thiol group and the cell penetrating peptide's leaving group during synthesis, specifically when double-stranded RNA is linked to transportan, pIs1, TAT(48-60), pVEC, MTS, MAP or Pep-1.

In certain embodiments the double-stranded RNA is operably linked to the cell penetrating peptide via a chemical linker. Examples of such linkages typically incorporate 1-30 nonhydrogen atoms selected from the group including C, N, O, S and P. Exemplary linkers include, but are not limited to, a substituted alkyl or a substituted cycloalkyl. Alternately, the heterologous moiety may be directly attached (where the linker is a single bond) to the amino or carboxy terminus of the cell-penetrating peptide. When the linker is not a single covalent bond, the linker may be any combination of stable chemical bonds, optionally including, single, double, triple or aromatic carbon-carbon bonds, as well as carbon-nitrogen bonds, nitrogen-nitrogen bonds, carbon-oxygen bonds, sulfur-sulfur bonds, carbon-sulfur bonds, phosphorus-oxygen bonds, phosphorus-nitrogen bonds, and nitrogen-platinum bonds. In some embodiments, the linker incorporates less than 20 nonhydrogen atoms and is composed of any combination of ether, thioether, urea, thiourea, amine, ester, carboxamide, sulfonamide, hydrazide bonds and aromatic or heteroaromatic bonds. In certain embodiments, the linker is a combination of single carbon-carbon bonds and carboxamide, sulfonamide or thioether bonds.

A general strategy for conjugation involves preparing the cell-penetrating peptide and the double-stranded RNA components separately, wherein each is modified or derivatized with appropriate reactive groups to allow for linkage between the two. The modified double-stranded RNA is then incubated together with a cell-penetrating peptide that is prepared for linkage, for a sufficient time (and under such appropriate conditions of temperature, pH, molar ratio, etc.) as to generate a covalent bond between the cell-penetrating peptide and the double-stranded RNA.

Numerous methods and strategies of conjugation will be readily apparent to one of ordinary skill in the art, as will the conditions required for efficient conjugation. By way of example only, one such strategy for conjugation is described below, although other techniques, such as the production of fusion proteins or the use of chemical linkers is within the scope of the present disclosure.

In certain embodiments, when generating a disulfide bond between the double stranded RNA and the cell-penetrating peptide of the present disclosure, the double-stranded RNA may be modified to contain a thiol group, and a nitropyridyl leaving group may be manufactured on a cysteine residue of the cell-penetrating peptide. Any suitable bond (e.g., thioester bonds, thioether bonds, carbamate bonds, etc.) may be created according to methods generally and well known in the art. Both the derivatized or modified cell-penetrating peptide, and the modified double-stranded RNA are reconstituted in RNase/DNase sterile water, and then added to each other in amounts appropriate for conjugation (e.g., equimolar amounts). The conjugation mixture is then incubated for 60 min at 37° C., and then stored at 4° C. Linkage may be checked by running the vector-linked double-stranded RNA molecule, and an aliquot that has been reduced with DTT, on a 15% non-denaturing PAGE. Double-stranded RNA molecules may then be visualized with the appropriate stain.

Methods of Administration

In certain embodiments, the cell permeable RNA inhibitor or conjugates of the present disclosure are formulated for intranasal delivery, for example as a nasal spray. In some other embodiments, the cell permeable RNA inhibitor or conjugates of the present disclosure are formulated for systemic delivery, for example as an intravenous injection or oral medication.

To facilitate delivery to a cell, tissue, or subject, the cell permeable RNA inhibitor, or conjugate thereof, of the present disclosure may, in various compositions, be formulated with a pharmaceutically-acceptable carrier, excipient, or diluent. The term “pharmaceutically-acceptable”, as used herein, means that the carrier, excipient, or diluent of choice does not adversely affect either the biological activity of the cell permeable RNA inhibitor or conjugate or the biological activity of the recipient of the composition. Suitable pharmaceutical carriers, excipients, and/or diluents for use in the present disclosure include, but are not limited to, lactose, sucrose, starch powder, talc powder, cellulose esters of alkonoic acids, magnesium stearate, magnesium oxide, crystalline cellulose, methyl cellulose, carboxymethyl cellulose, gelatin, glycerin, sodium alginate, gum arabic, acacia gum, sodium and calcium salts of phosphoric and sulfuric acids, polyvinylpyrrolidone and/or polyvinyl alcohol, saline, and water.

In accordance with the methods of the present disclosure, the quantity of the double-stranded RNA or conjugate thereof that is administered to a cell, tissue, or subject should be an effective amount.

The following examples show that Pen1 may be used to intranasally deliver siRNA to a patient in an effective amount. The siRNA used in the examples may be replaced with other siRNAs, particularly other p75NTR inhibitors, in similar treatment methods. Further, other types of double stranded RNAs, particularly other types of p75NTR inhibitors, may also be used in lieu of the siRNA in the example. Additionally, other cell penetrating peptides, as described above more fully, that act similarly to Pen1 may be used instead of Pen1. In general all aspects of the examples may be combined with other aspects and embodiments of the present disclosure.

For other diseases or indications, an appropriate and effective double stranded RNA and cell penetrating peptide may be chosen based on the disease and the patient that has the disease.

### EXAMPLES

The following examples are provided to, in part, demonstrate the effects of one siRNA-cell penetrating peptide construct according to an embodiment of the disclosure. This embodiment need not include every detail presented in these examples in order to function and may, in particular, differ in the exact construct used and is dose administered and dosing regimen. In addition, the embodiment of these examples is clearly intended for adaptation to traumatic brain injuries in humans, which have different causes than the induced brain injuries in mice.

### Example 1: CCI Mouse Model and Treatment

All animal studies were conducted using the National Institutes of Health guidelines for the ethical treatment of animals with approval of the Rutgers Animal Care and Facilities. Adult Mice (C57BI/6, RRID:MGI:5656552) between the ages of 2 and 3 months were maintained on a 12 hour light/dark cycle with free access to food and water.

Male mice at 10-12 weeks old were subjected to a CCI injury. Animals were handled one day before and on the day after the CCI to reduce the effects that stress might have on the behavioral tests. The animals were anesthetized with a mixture of ketamine (90 mg/kg) and xylazine (10 mg/kg) i.p. Once fully anesthetized, the scalp was cleansed and an incision along the midline was created to expose the skull. The animals were placed in a stereotaxic frame (David Kopf Instruments, Tujunga CA). A 3-mm craniectomy was produced using a trephine midway between Bregma and Lambda, 2.5 mm lateral to the sagittal suture (somatosensory cortex). The brain injury was generated using a 3 mm diameter impactor tip. The velocity of the impactor was set at 4.0 m/s, depth of penetration was 1.5 mm and the duration of deformation was 150 ms. Animals were randomly assigned to receive either a sham injury or brain injury. The animals were placed on heating pads at 37° and monitored continuously for 2 hours after surgery. Buprenorphine (0.05 mg/kg, SC) was administered post-operatively. Additionally, all animals received 3% body weight of 0.9% saline subcutaneously to prevent dehydration.

To assess whether acutely blocking the induction of p75NTR that occurs following injury would provide neuroprotection, administered an siRNA directed against p75NTR was administered immediately following the injury to some of the mice. siRNA directed against the p75NTR sequence (sense, SSUGGAACAGCUGCAAACAAAUU, (SEQ ID NO. 1).) or luciferase sequence (sense, SSCGUACGCGGAAUACUUCGAUU, SEQ ID NO. 13) was synthesized (Dharmacon, Thermo Fisher Scientific) and linked to Penetratin-1. Two μl drops of 80 nM p75NTR siRNA or Luciferase siRNA (control siRNA) were administered under anesthesia to each nostril every 2 min for a total of 20 μl. Animals were randomly assigned to each treatment. Control animals received purified rabbit IgG antibody (BD Biosciences Cat #550875, RRID:AB_393942).

### Example 2: Immunohistochemistry and TUNEL Staining Demonstrate p75NTR in Dying Brain Tissue in CC Model Mice

The p75NTR is induced after multiple different types of injury to the CNS, including seizures, spinal cord injury, and corticospinal transection. The neurons that show induction of p75NTR in those injury paradigms are apoptotic, and p75NTR was shown to mediate neuronal death in response to proneurotrophin ligands in several of these injury conditions. To assess whether p75NTR and its ligands might also play a role in mediating neuronal death following TBI, the CCI model with a focal injury as described above was used in mice. p75NTR expression was examined during the subacute period of recovery following the injury. Sham animals that had been anesthetized and subjected to the craniotomy were used as controls.

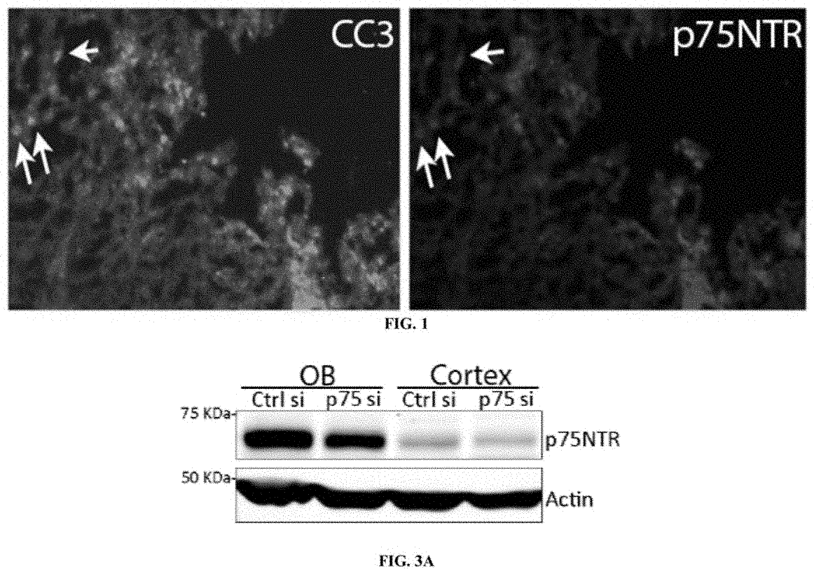

Animals were deeply anesthetized with ketamine/xylazine and perfused transcardially with saline followed by 4% paraformaldehyde. The brains were removed and post-fixed in 4% paraformaldehyde for two hours and cryoprotected in 30% sucrose. Sections (20 μm) were cut on a cryostat (Leica) and mounted onto charged slides. Sections were blocked in 1% BSA/5% donkey serum and permeabilized with PBS/0.3% Triton X-100, and then exposed to primary antibodies overnight at 4° C. in PBS/1% BSA. Slides were then washed three times in PBS, exposed to secondary antibodies coupled to different fluorophores at room temperature for one hour. Sections were washed again three times, with DAPI (4′,6′-diamidino-2-phenylindole; Sigma; 1:10,000) present in the final wash. Sections were coverslipped with antifading medium (ProLong Gold; Invitrogen) and analyzed by fluorescence (Nikon Eclipse TE200) and confocal microscopy (Zeiss LSM 510 META). Primary antibodies used were: anti-NeuN (1:500; Cell Signaling Technology Cat #12943, RRID:AB_2630395), anti-proBDNF (1:500; Alomone Labs Cat #ANT-006. RRID:AB_2039758), anti-GFAP (1:500; R&D Systems Cat #AF2594, RRID:AB_2109656) and anti-p75NTR (1:500; R&D Systems Cat #AF1157, RRID:AB_2298561). FIG. 1 shows representative immunofluorescent images of the results.

Following CCI injury, p75NTR expression was induced in the penumbral area adjacent to the injury, confirming results from previous studies. In particular, FIG. 1 shows that CC3, a marker of dying cells, co-localizes with p75NTR, demonstrating that cells expressing p75NTR were, in fact, inured and dying.

The number of apoptotic cells following TBI was assessed by labeling with terminal deoxynucleotidyl transferase-dUTP nick end according to the manufacture's protocol (click-iT TUNEL assay, Thermo Fisher, Cat #C10617). Sections were then immunostained for p75NTR and counterstained with DAPI TUNEL positive cells were analyzed on a Zeiss spinning disk confocal microscope using the tiling function to measure 10 fields of view of the lesion site and surrounding tissue. Quantification of TUNEL positive cells was made using Image J Version 1.51 (National Institutes of Health, USA).

The images from the TUNEL staining are shown in FIG. 2.

At 1 and 3 days after the injury, cells with high levels of p75NTR are also labeled with TUNEL (FIG. 2), supporting the conclusion that cells expressing p75NTR were undergoing cell death. In particular, in FIG. 2, arrowheads show co-localization of TUNEL and p75NTR in the mice subjected to CCI, while the Sham mice did have induction of TUNEL or p75NTR.

### Example 3: Western Blot Analysis of CCI Mice

One day after infusion with siRNA or the control, the OB and cortex were analyzed for p75NTR levels. Tissue from the OB and cortex were dissected and homogenized using 1% NP40, 1% triton, 10% glycerol in TBS buffer (50 mM Tris, pH 7.6, 150 mM NaCl) with protease inhibitor cocktail (Sigma Aldrich, St. Louis, Mo., USA). The protein lysates were sonicated and centrifuged for 15 minutes at 4° C. Proteins were quantified using the Bradford assay (Bio-Rad, Cat #500-006) and equal amounts of protein were loaded onto SDS gels and transferred to nitrocellulose membranes.

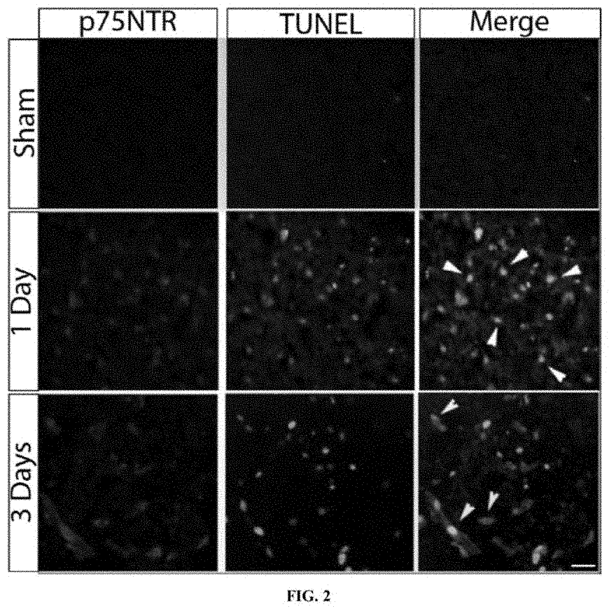

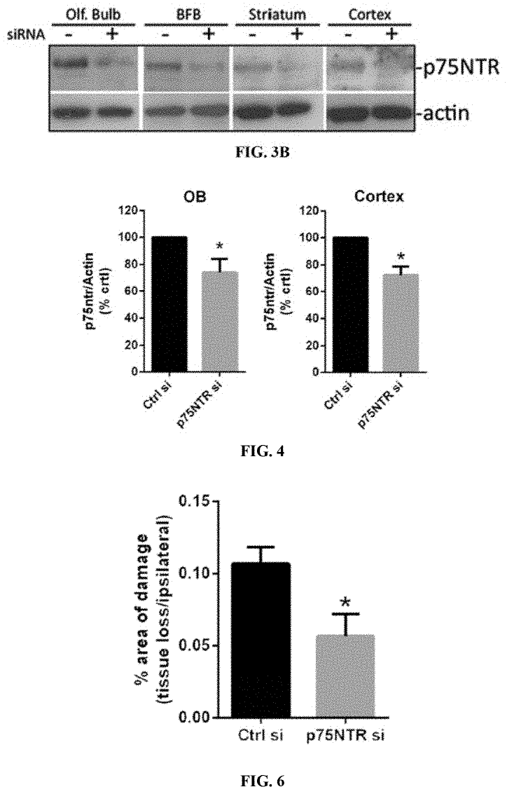

Membranes were blocked in 5% nonfat dried skim milk in TBS-T for 2 h at RT. Primary antibody (anti-p75NTR, Millipore Cat #07-476, RRID:AB_310649) diluted 1:1000 in 1% BSA was applied overnight at 4° C. Membranes were washed with TBS-T 3×10 min each and incubated with secondary anti-rabbit horseradish peroxidase (HRP)-conjugated IgG antibody for 1 h at RT (Jackson ImmunoResearch, West Grove, Pa., USA). To confirm equal protein levels, blots were re-probed for actin (Sigma, cat #A5316, RRID: AB 476743). Bands were scanned with the Odyssey infrared imaging system (LI-COR Bioscience) and quantified using ImageJ version 1.52e (National Institutes of Health, USA). FIG. 3A is a representative western blot and FIG. 4A is a graph of the western blot results. Both brain regions analyzed showed reduced p75NTR levels in the animals that received the p75NTR Pen-siRNA infusion compared with the control infusion (p<0.05).

A duplicate test was performed with an additional cohort of mice to determine whether differences were seen in the effects on p75NTR expression depending on brain region. In particular, samples were taken from the BFB and striatum of the mice, in addition to the OB and cortex. Samples were processed as described above with respect to FIG. 3A and Western Blot results are presented in FIG. 3B. A decrease in p75NTR was also seen in the additional brain regions. [is this a correct assessment of the data?]

### Example 4: Determining the Area of Damage in CCI Model Mice

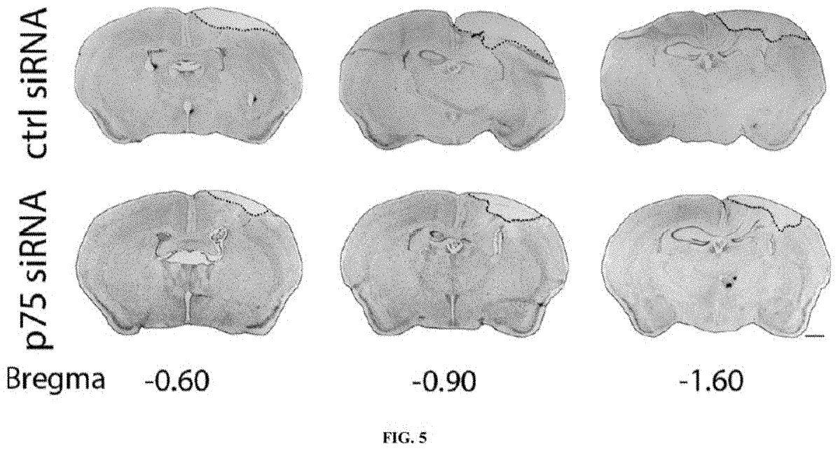

p75NTR Pen-siRNA or luciferase Pen-siRNA was applied intranasally to mice immediately following the CCI injury, and the mice were allowed to recover for 2 to 3 days. A total of 12 sections through the injured cortex (Bregma −0.3 mm to −1.80 mm) were selected (20 μm thickness, spaced every 200 μm). The brain tissue sections were stained with Cresyl Violet and coverslipped with Permount mounting media or stained for NeuN and coverslipped with antifading medium with DAPI (ProLong Gold with DAPI, Thermo Fisher Cat #P36931). The area of tissue loss in the injured hemisphere was traced using the contralateral hemisphere superimposed on top of the lesioned hemisphere. The area that had been damaged in the ipsilateral hemisphere, which included the area of tissue loss and the penumbra, was quantified and divided by the total area of the ipsilateral hemisphere. Area measurements were obtained from at least 3 animals per group using Image J Version 1.51. FIG. 5 is a collection of representative images of the stained coronal sections, and FIG. 6 is a graph that quantifies those images.

Morphological analyses of sections stained with cresyl violet revealed that the mice that received the p75NTR Pen1-siRNA showed a significant reduction in neocortical of damage compared with the mice that received Pen1-siRNA control.

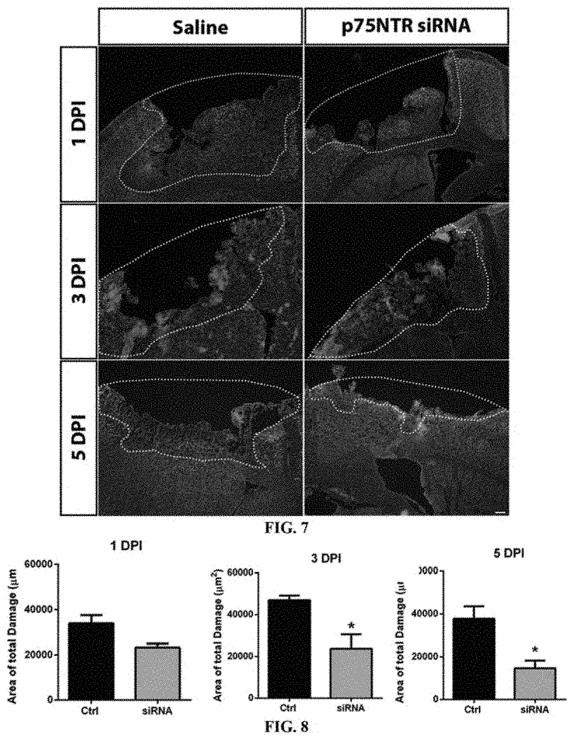

A different cohort of mice were treated with either saline or p75NTR siRNA immediately after CC1. After 1 day, 3 days, or 5 days post infusion, brain sections were taken and stained for NeuN and counterstained with DAPI to reveal the area of damage, as illustrated in FIG. 7. The area of total damage comprised of the area of tissue loss and the penumbra (dotted line), where the density of DAPI and NeuN staining was reduced. Results are quantified in FIG. 8. The percentage of the total area of damage relative to the saline control was significantly reduced by the p75NTR siRNA on all three days, demonstrating lasting protective effects of p75NTR siRNA.

### Example 5: Modified Neurological Severity Score and Movement Testing

Prior to perfusion, the mice were analyzed using a series of tests to determine whether the pen-siRNA provided behavioral as well as morphological sparing.

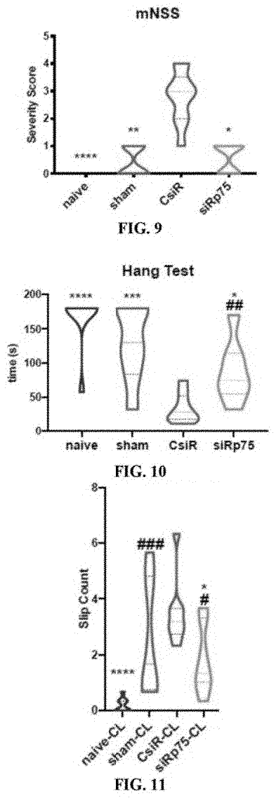

Animals were analyzed using a battery of tests to assess motor, balance, sensory, exploratory, and reflex behaviors that make up the mNSS. Successful completion of each task results in a “0” score while failure results in a “1” score. Scores for each task are added to create a total composite score out of 12. High final mNSS scores were indicative of task failures and interpreted as neurological impairment. The mNSS test is outlined further in the following references: Flierl M. A., Stahel P. F., Beauchamp K. M., Morgan S. J., Smith W. R., Shohami E. (200)) Mouse closed head injury model induced by a weight-drop device. Nat. Protoc. 4, 1328-1337; Chen J. L., Sanberg P. R., Li Y., Wang L., Lu M., Willing A. E., Sanchez-Ramos J., Chopp M. (2001) Intravenous administration of human umbilical cord blood reduces behavioral deficits after stroke in rats. Stroke 32, 2682-2688; Wu W., Chen X., Hu C., Li J., Yu Z., Cai W. (2010) Transplantation of neural stem cells expressing hypoxia-inducible factor-1α (HIF-1α) improves behavioral recovery in a rat stroke model. J. Clin. Neurosci. 17, 92-95. FIG. 9 summarizes the results of the mNSS test for the four mice groups.

Mice that sustained a CCI and had received p75NTR Pen1-siRNA showed significantly preserved sensorimotor function two days after surgery compared to the CCI group that was given control Pen1-siRNA. On the mNSS test, p75NTR Pent-siRNA treated mice consistently scored lower than control Pen1-siRNA treated mice and were comparable to sham operated mice.

Mice were also evaluated for their abilities in a hang test. For the hang test, mice were allowed to grab onto a thin, elevated, horizontal metal rod by their forelimbs. The length of time that the mouse spent on the metal rod without falling was measured. A maximum time of 3 minutes on the rod was allotted per trial. Mice were tested 3 times consecutively. Results are shown in FIG. 10. The p75NTR Pen1-siRNA treated mice showed some muscle weakness (as reflected by short durations hanging onto the rod) when compared to the naïve animals, but their performance was significantly better than the control Pen1-siRNA group.

A horizontal ladder test was used to evaluate injury to the sensorimotor cortex. A ladder with 4 mm diameter rungs were irregularly spaced, with a minimum spacing of 12 mm and a maximum spacing of 24 mm, was used. The ladder was suspended horizontally 18 inches above the ground. One end contained a hollow black goal box where a sugar rich cereal treat was placed. Rungs are suspended along an 8 cm wide beam. A video camera was placed directly in front of the apparatus and a mirror was situated below the apparatus so that foot-slips were readily visible. Training consisted of a 5-minute acclimation period in the goal box, followed by at least three trials where the animal was directed to run across the ladder beam towards the goal box. On the testing day, each animal had to complete three runs where they completely traverse the ladder at a constant rate without turning around. Between each test run the animal was left in the goal box for 1 min. A foot-slip was scored when either of the limbs dropped below the plane of the rungs due to misplacement on either the rung ahead or behind. The number of ipsilateral and contralateral forelimb and hindlimb foot-slips were counted. Results of the horizontal ladder test are in FIG. 11.

The CCI injured mice treated with the control Pen1-siRNA made foot-faults when using their limbs contralateral to the CCI, whereas they made few foot-faults using their limbs ipsilateral to the lesion. The p75NTR Pen1-siRNA treated mice had fewer foot faults than control Pen1-siRNA treated mice (p<0.05). There was no significant difference in ipsilateral foot slips between groups indicating the specificity of both injury and recovery of sensorimotor function after treatment.

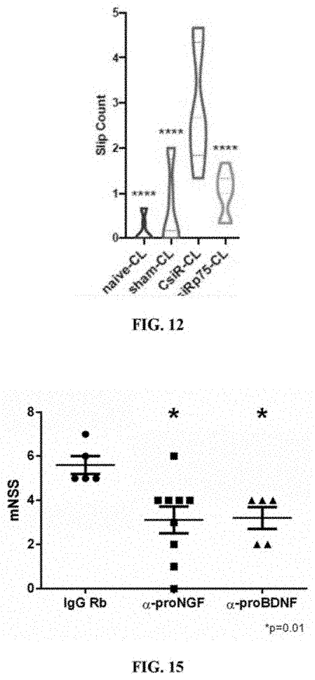

Foot slips were also measured on horizontal beam walk test as part of the mNSS battery. The results for the beam walking test are in FIG. 12. p75NTR Pen1-siRNA treated mice had significantly fewer contralateral foot slips (p<0.001) than control Pen1-siRNA treated mice on a 1.0 cm horizontal beams. The mice groups were also assessed on 0.7 cm and 1.5 cm horizontal beams. p75NTR Pent-siRNA treated mice exhibited significant improvement over control Pen1-siRNA mice on both of those beams, but the differences did not reach statistical significance.

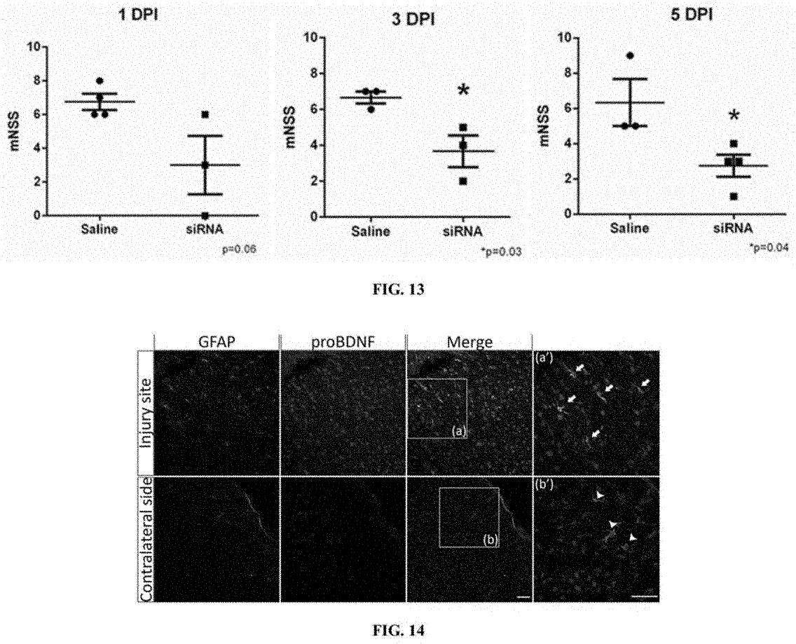

A separate cohort of mice treated in a similar manner, but with a saline treatment control, were assessed for mNSS one day, three days, and 5 days after treatment. Results are presented in FIG. 13. These mice exhibited consistent mNSS scores over time that were also consistently better than in the control mice, demonstrating durable protective effects of p75NTR Pen1-siRNA treatment.

### Example 6: Further Confirmation of the Role of p75NTR in Neuronal Cell Death

Studies have that the levels of proNGF increases after TBI in astrocytes and microglia. To assess whether proBDNF was also upregulated after TBI, mice were subjected to CCI and perfused 3 days after injury. Representative sections through the injury site 3 days after CCI, presented in FIG. 14 show increased proBDNF labeling (red), some of which colocalizes with GFAP (green) adjacent to the area of tissue damage. proBDNF was induced in GFAP-expressing astrocytes as well as in other cell types following TBT. In contrast, sections through the contralateral side 3 days after the injury show little expression of proBDNF.

Whether inhibition of these proneurotrophin ligands that activate p75NTR would prevent neuronal death and functional loss after TBI was also analyzed. Neutralizing antibodies to either proNGF or proBDNF infused intranasally to each nostril every 2 min for a total of 20 μl to the mice intranasally immediately following the CCI injury. Controls received an equal amount of pre-immune IgG. Two days following the injury, sensorimotor function was analyzed using the mNSS test battery. Data were collected across 5 to 8 animals per group and results are presented in FIG. 15. The neutralizing antibodies to either proNGF or proBDNF provided significant functional sparing compared with control IgG as assessed by the mNSS.

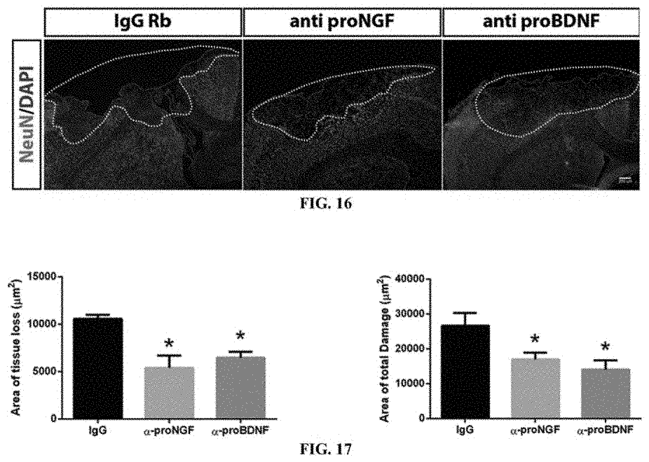

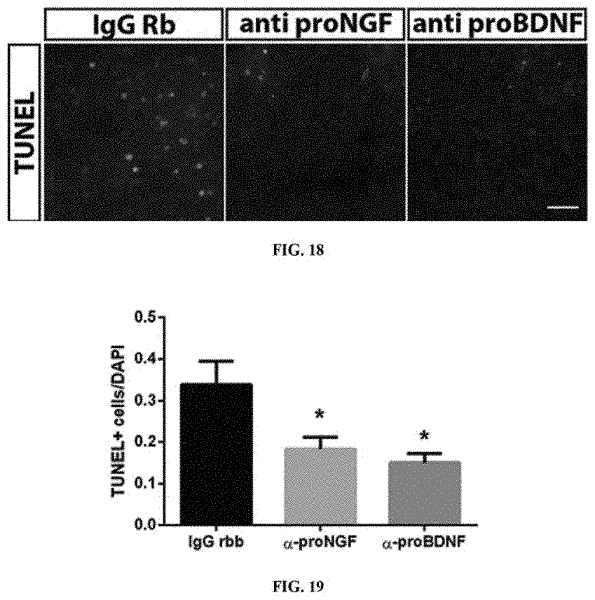

When the mice were perfused 3 days after treatment, morphological analysis of the brains from the animals that had received the blocking antibodies to proNGF or proBDNF showed that the area of total damage (the area of tissue loss and the penumbra) was reduced by the application of the antibodies to either ligand (see FIG. 16 and FIG. 17.) Moreover, the number of TUNEL-positive cells in the penumbra was reduced by 50% following administration of the proneurotrophin antibodies as shown in FIG. 18 and FIG. 19. These data demonstrate that the proneurotrophin-p75NTR pathway contributes to delayed cell death following TBI and that either preventing induction of the receptor or blocking the ligands can provide neuroprotection and rescue sensorimotor function.

The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims.

Patents, patent applications, publications, procedures, and the like are cited throughout this application, the disclosures of which are incorporated herein by reference in their entireties.

This application claims the benefit of priority to U.S. Provisional Application No. 63/013,779, filed Apr. 22, 2020, which application is hereby incorporated by reference in its entirety. Various publications mentioned herein are also incorporated by reference herein in their entirety.

1. A method for the intranasal delivery of RNA comprising administering, by intranasal delivery, an effective amount of cell-permeable RNA inhibitor effective to treat a disease of a patient,
wherein the cell-permeable RNA inhibitor comprises (i) a double stranded RNA which is effective in inhibiting the expression of the target protein operably linked to (ii) a cell-penetrating peptide.

2. The method of claim 1, wherein the cell-penetrating peptide is Penetratin1.

3. The method of claim 1, wherein the cell-penetrating peptide is selected from the group including transportan, pIS1, Tat(48-60), pVEC, MAP, Pep-1, and MTS.

4. The method of claim 2, wherein the cell-permeable RNA inhibitor is administered at a concentration between 1 nM and 1,000 nM, inclusive.

5. The method of claim 2, wherein a p75NTR siRNA inhibitor is conjugated to the cell-penetrating peptide via a disulfide bond.

6. The method of claim 5, wherein the p75NTR siRNA inhibitor is administered at a concentration between 1 nM and 500 nM, inclusive.

7. The method of claim 5, wherein concentration of the p75NTR siRNA inhibitor conjugated to the cell-penetrating peptide treats the traumatic brain injury by decreasing apoptosis in the patient's brain.

8. The method of claim 5, wherein the concentration of the p75NTR siRNA inhibitor conjugated to the cell-penetrating peptide treats the traumatic brain injury by decreasing an amount of p75NTR the patient's brain.

9. The method of claim 2, wherein the double stranded RNA is a small interfering RNA.

10. The method of claim 2, wherein the double-stranded RNA is selected from a group including small temporal RNA, small nuclear RNA, small nucleolar RNA, short hairpin RNA and microRNA.

11. The method of claim 1, wherein the double stranded RNA is further attached to a label selected from the group including an enzymatic label, a chemical label, and a radioactive label.

12. The method of claim 2, wherein a double stranded RNA is conjugated to the cell-penetrating peptide via a disulfide bond.

13. A composition for administration into the central nervous system or pulmonary system comprising a solution including effective amounts of:
(i) a cell-permeable RNA inhibitor and
(ii) an excipient,
wherein the cell-permeable RNA inhibitor comprises a double stranded RNA effective in inhibiting the expression of a target protein encoded by a target mRNA operably linked to a cell penetrating peptide.

14. The composition of claim 13, wherein the double-stranded RNA is a small interfering RNA.

15. The composition of claim 13, wherein the double-stranded RNA is selected from the group including a small temporal RNA, small nuclear RNA, small nucleolar RNA, short hairpin RNA and microRNA.

16. The composition of claim 13, wherein the cell-penetrating peptide is selected from the group including Penetratin1, transportan, pIS1, Tat(48-60), pVEC, MAP, Pep-1 and MTS.

17. The composition of claim 13, wherein a p75NTR siRNA inhibitor is conjugated to the cell-penetrating peptide via a disulfide bond.


SEQUENCE LISTING (13 sequences)

SEQ ID NO: 1 - RNA, 23 nt, Artificial Sequence
  p75NTR siRNA inhibitor sequence
ssuggaacagcugcaaacaaauu

SEQ ID NO: 2 - protein, 18 aa, Artificial Sequence
  Synthetic peptide sequence
  MOD_RES 2: Pys
CNRQIKIWFQNRRMKWKK

SEQ ID NO: 3 - protein, 16 aa, Artificial Sequence
  Exemplary cell permeant molecules RL16
RRLRRLLRRLLRRLRR

SEQ ID NO: 4 - protein, 12 aa, Artificial Sequence
  Sequence derived from Penetratin1 with slightly different physical properties
RVGRRRRRRRRR

SEQ ID NO: 5 - protein, 27 aa, Artificial Sequence
  Synthetic sequence
GWTLNSAGYLLGKINLKALAALAKKIL

SEQ ID NO: 6 - protein, 16 aa, Artificial Sequence
  Synthetic sequence
PVIRVWFQNKRCKDKK

SEQ ID NO: 7 - protein, 13 aa, Artificial Sequence
  Synthetic sequence
GRKKRRQRRRPPQ

SEQ ID NO: 8 - protein, 16 aa, Artificial Sequence
  Synthetic sequence
LLIILRRRIRKQAHAH

SEQ ID NO: 9 - protein, 27 aa, Artificial Sequence
  Synthetic sequence
GALFLGWLGAAGSTMGAWSQPKKKRKV

SEQ ID NO: 10 - protein, 18 aa, Artificial Sequence
  Exemplary MAP includes the amino acid sequence
KLALKLALKALKAALKLA

SEQ ID NO: 11 - protein, 21 aa, Artificial Sequence
  Relevant amphipathic peptide is Pep-1
KETWWETWWTEWSQPKKKRKV

SEQ ID NO: 12 - protein, 21 aa, Artificial Sequence
  Synthetic sequence
KETWWETWWTEWSQPKKKRKV

SEQ ID NO: 13 - RNA, 23 nt, Artificial Sequence
  Luciferase sequence
sscguacgcggaauacuucgauu